(19) United States
(12) Patent Application Publication
(10) Pub. No.: US 2019/0269530 A1
(43) Pub. Date: Sep. 5, 2019

(54) LINER FOR A PROSTHESIS, AND PROSTHESIS

(71) Applicant: OTTOBOCK SE & CO. KGAA, Duderstadt (DE)

(72) Inventors: Luder MOSLER, Duderstadt (DE); Christian MULLER, Kalefeld (DE); Thomas KETTWIG, Luedersdorf Ot Herrnburg (DE)

(73) Assignee: OTTOBOCK SE & CO. KGAA, Duderstadt (DE)

(21) Appl. No.: 16/417,288

(22) Filed: May 20, 2019

Related U.S. Application Data
(63) Continuation of application No. 14/425,593, filed on Mar. 3, 2015, now abandoned.

(30) Foreign Application Priority Data
Sep. 3, 2012 (DE) 102012017324.5

(51) Int. Cl. A61F 2/78; A61F 2/80

(52) U.S. Cl. CPC A61F 2/7812; A61F 2002/7818; A61F 2002/7875; A61F 2/80

(57) ABSTRACT

The invention relates to a liner (16) for a prosthesis, which liner (16) has a proximal opening, for receiving an amputation stump, and a distal end lying opposite the proximal opening, and it is produced from a liner material (4), wherein a longitudinal direction of the liner (16) extends from the proximal opening to the distal end, and a circumferential direction runs perpendicular to the longitudinal direction, wherein the liner (16) has a plurality of fibres (6), which are made from an inelastic material and are arranged in such a way that a lengthening of the liner (16) in the longitudinal direction results in a shortening of the liner (16) in the circumferential direction, and vice versa, wherein the liner (16) has non-slip means (26) which, when the liner (16) is fitted in place, prevent the liner (16) from slipping on the amputation stump.

The invention relates to a liner for a prosthesis, said liner having a proximal opening for receiving an amputation stump and a distal end, and being produced from a liner material, wherein a longitudinal direction of the liner extends from the proximal opening to the distal end and a circumferential direction extends perpendicularly to the longitudinal direction. The invention also relates to a prosthesis having such a liner and to a prosthesis socket for such a prosthesis.

Liners of the generic type and prostheses equipped therewith have been known for a long time from the prior art. The prosthesis has a prosthesis socket which has usually been produced from a rigid material, for example a fiber-reinforced synthetic material. This material is easy to process, is stable and nevertheless has a low intrinsic weight such that it is highly suitable for components of a prosthesis. In order to avoid contact between the skin of the amputation stump of the patient and the rigid prosthesis socket and at the same time to provide cushioning, a liner made of a liner material is first of all pulled over the amputation stump before the amputation stump is introduced into the prosthesis socket. A suitable liner material is for example silicone. The liner material molds itself to the shape of the amputation stump in this case and therefore has an optimal fit and ensures an optimal seat.

The prosthesis additionally has a prosthetic device which is arranged on the prosthesis socket and comprises, inter alia, an artificial limb, for example an artificial lower leg with a foot arranged thereon. Parts of the prosthetic device are additionally fastening and locking means, and optionally pump devices, valves and the like, necessary for the operation of the prosthesis.

Subsequently, the prosthesis socket is arranged on the liner. Conventionally, the prosthesis socket consists entirely of the rigid material. The amputation stump is consequently located in a shell which separates it completely from the surroundings. In particular, it is not possible for the wearer of the prosthesis to perceive impressions of the environment, for example contact with the amputation stump, via the amputation stump. In addition, as a result of the complete sheathing of the liner, which is located on the amputation stump, the breathability of the liner material is considerably reduced and limited. Both of these result in an uncomfortable and artificial wearing feel and in reduced wearing comfort of the prosthesis and the liner.

However, in particular in the case of leg prostheses, frame-type sockets, which are interrupted at at least one location and thus have at least one window, have the disadvantage that, on account of the very high loading of the amputation stump when walking with the prosthesis, the amputation stump covered with the liner can spill out of the windows in the frame-type socket. This can result in pressure points and also, as a result of rubbing in particular at the peripheries of the windows in the frame-type socket, in sometimes painful wounds, in particular in the form of what is referred to as window edema.

In addition, in the case of a frame-type socket, it is of course not possible to secure the frame-type socket to the amputation stump, or the liner pulled over the latter, by the generation of a negative pressure between the liner and the liner prosthesis socket. Since the frame-type socket has at least one interruption in the form of the window, a closed volume which could be subjected to a negative pressure does not exist here. Therefore, frame-type sockets are conventionally fastened to liners via a fastening device provided at the distal end of the liner. As a result, the tensile forces that occur in particular during the swing phase of walking with the prosthesis act only on the distal end of the liner, with the result that what is referred to as the milking effect can occur. Over time, for example when the prosthesis is worn for an entire day, this results in a change in volume and a change in shape of the amputation stump. The liner material has to be flexible enough to be able to follow this change in shape and volume of the amputation stump, in order to ensure a firm seat of the prosthesis on the amputation stump of the patient. However, as a result of a particularly flexible liner material that is required therefor, it is easier for the liner, or the amputation stump covered therewith, to spill out of the windows in the frame-type socket.

Therefore, the invention is based on the object of proposing a liner and a prosthesis equipped therewith, by way of which a secure hold of the liner and of the prosthesis on the amputation stump of the patient is ensured even over a long period of time, and with which spilling of the amputation stump out of the windows of a frame-type socket is effectively prevented at the same time.

The invention achieves the stated object by way of a liner of the generic type according to the preamble of claim 1, said liner being distinguished by the fact that the liner has a plurality of fibers which consist of a nonelastic material and are arranged such that an elongation of the liner in the longitudinal direction results in shortening of the liner in the circumferential direction and vice versa, wherein the liner has anti-slip means by way of which the liner is prevented from slipping on the amputation stump in the fitted state of the liner.

Advantageously, the fibers are in this case arranged such that elastic deformation of the liner is possible both in the longitudinal direction and in the circumferential direction. However, this is not necessarily the case. All that is important is that when the liner is pulled on, that is to say when the amputation stump is introduced into the liner, the liner can optimally adapt to the shape of the amputation stump. This generally occurs in that the liner is rolled up from the distal end of the amputation stump and thus is brought into contact with the amputation stump over a large area.

Advantageously, the fibers are in this case embedded in the liner material. This has the result that there are no disturbing pressure points or irregularities on the liner which can result in discomfort or even pain.

Consequently, the liner can adapt optimally to the particular contour of the amputation stump both in the circumferential direction and in the longitudinal direction, in particular when elastic deformation is possible in both directions. During a day, the volume of the amputation stump changes. If its circumference decreases, for example, the liner would also have to become shorter in the circumferential direction in order to continue to ensure an optimal seat. However, this would result in elongation of the liner in the longitudinal direction. This means that a large proportion of the inner surface of the liner which is in contact with the skin of the wearer would have to move relative to the skin. However, on account of the anti-slip means, this is not possible and so the liner does not follow the change in volume. This is advantageous in particular when the volume of the amputation stump increases greatly with the result that the stump can spill out of windows in the prosthesis socket. The liner would have to expand in the circumferential direction; however, this would result in shortening in the longitudinal direction, and is therefore not possible on account of the anti-slip means.

If the liner is intended to be adapted to the changed volume of the amputation stump, the liner merely has to be removed from the amputation stump and pulled over it again. In this case, the liner can adapt optimally again to the present shape of the amputation stump.

Elastic deformation of the liner in one direction consequently always results in deformation in the other direction, this being associated with extensive shifting of the liner material relative to the amputation stump of the patient and therefore not being possible in the fitted state of the liner.

In a preferred embodiment, the anti-slip means are a surface property of the inner side of the liner, said inner side resting against the amputation stump in the fitted state of the liner. As a result of this surface property, increased adhesive friction or adhesion between the inner surface of the liner and the skin of the wearer of the liner occurs, such that in this case, on account of the increased adhesive friction or adhesion, extensive shifting and slipping off the liner on the amputation stump, as would be necessary for a change in volume, cannot take place.

In a preferred configuration of the liner, the inner surface thereof, which comes into contact with the skin of the wearer of the prosthesis in the fitted state, is provided with a coating which improves adhesive friction between the liner and the skin of the wearer and thus enhances the desired effect. Alternatively or in addition thereto, provision can also be made of a separate additional liner which is pulled over the actual liner and has increased stability in the longitudinal direction in order to be able to absorb forces in the longitudinal direction.

Alternatively or in addition thereto, the anti-slip means can have at least one enclosing element which is designed to enclose the amputation stump in the fitted state such that the liner is prevented from slipping. The enclosing element can in this case be for example a sock-like or sleeve-like element which is pulled over the liner after the liner has been arranged on the amputation stump, and is arranged for example in the proximal region of the liner. The enclosing element in this case exerts a pressure on the liner over the circumference of the amputation stump on which the liner is located, and in this way ensures that increased adhesive friction between the inner surface of the liner and the amputation stump occurs in this region. In this way, too, it is possible, for example without a surface coating on the inner side of the liner, to achieve the desired adhesive friction in order to prevent the liner from slipping on the amputation stump. Depending on the required strength of the adhesive friction and the adhesive friction already achieved in some other way, the width of the enclosing element can vary in the longitudinal direction of the liner. If only a small increase in the adhesive friction is required by the enclosing element, the latter can be formed in a correspondingly narrow manner, while it should be formed in a wide manner if a large increase in the adhesive friction between the inner side of the liner and the amputation stump is intended to be achieved.

The enclosing element does not in this case have to enclose the amputation stump completely, that is to say around the entire circumference thereof. Partial enclosing can also be sufficient to apply the necessary forces to the liner and the amputation stump.

The enclosing element can also consist for example in the form of a synthetic component, for example made of a plastic material, which has two tongue-like protrusions which are placed around the amputation stump. The two tongue-like protrusions are connectable together via a fastening element, wherein in this way the pressure required to prevent the liner from slipping is applied. In particular, the circumference enclosed by the two tongue-like protrusions can advantageously be adjusted in a continuously variable manner such that the force applied to the amputation stump can also be adapted optimally to the requirements of the wearer. For the purpose of closing, for example a strap- or belt-like element can be arranged on one of the two tongues, said element being fastenable to the respectively other tongue-like protrusion for example via touch-and-close elements. Of course, it is also possible for the element to be able to be pulled through a loop and be fastened to itself likewise via touch-and-close elements.

In another configuration, the synthetic element is configured such that when the prosthesis is fitted on the amputation stump, it is expanded and thus elastically pretensioned. As a result, a clamping force is applied to the liner arranged on the amputation stump, said clamping force likewise preventing the liner from slipping on the stump.

Advantageously, the plurality of fibers has at least a first fiber and at least a second fiber which are arranged such that they cross one another preferably at a right angle. This relates to the liner in the state in which it is not extended in any direction or elastically deformed. It has been found to be particularly advantageous when the first fiber and the second fiber form a rhombic pattern in which the internal angles of each rhombus are right angles and the fibers enclose an angle of about 45° or −45° with the longitudinal direction of the liner.

Since at least one first fiber and at least one second fiber are provided in the liner material, said fibers being arranged such that they cross one another, a volume-stable liner is produced. If a liner according to the invention is pulled over an amputation stump, on account of the flexibility and elasticity of the liner material, it adapts optimally to the amputation stump. Conventionally, such a liner is rolled onto the amputation stump and so the liner according to the invention can be fitted in the same way as a conventional liner. The first fibers and second fibers embedded in the liner material arrange themselves in this case such that they optimally follow the shape of the amputation stump. When the liner is fitted on the amputation stump, the at least one first fiber and the at least one second fiber also follow the external contour of the amputation stump such that an angle at which the two fibers cross is set freely. Limits to this settability of the angle are only given by the liner material in which the first fibers and the second fibers are embedded.

In a preferred embodiment, the first fiber forms a spiral matrix in a first circumferential direction and the second fiber forms a spiral matrix in a second circumferential direction opposite to the first circumferential direction. Spiral means in this case that each fiber extends around the circumference of the amputation stump preferably through a number of turns when the liner rests against an amputation stump, and in this case extends for example from the distal end of the liner to the proximal opening. Since the first fiber and the second fiber extend in different circumferential directions about the amputation stump, when the liner is fitted the two fibers cross at a large number of different locations. In this way, the desired volume stability is achieved and at the same time optimal shape adaptation of the liner when it is fitted on the amputation stump is ensured.

Preferably, the first fiber is part of a first fiber layer made of a plurality of first fibers and the second fiber is part of a second fiber layer made of a plurality of second fibers. The individual fibers of each fiber layer can in this case extend preferably parallel to one another. In this configuration, use can be made of extensive fiber layers which can be cut to the optimal size for the liner desired in each case.

Preferably, the liner has at least two part-liners, wherein the at least one first fiber is embedded in a first part-liner and the at least one second fiber is embedded in a second part-liner, and the second part-liner is intended to be pulled over an amputation stump after the first part-liner has been pulled over the amputation stump. The at least two part-liners are consequently both configured to be pulled over the amputation stump. They each consist of a liner material into which in each case only one of the provided fiber types is embedded. As a result of the at least two part-liners being pulled one over the other, the desired superimposition of the at least two fibers occurs such that they cross at an angle. It is particularly clear from this example that, in the context of the present invention, it is not necessary for the different fibers to be in contact on crossing. All that is important is that they extend in different directions and that they allow elastic deformation of the liner in the longitudinal direction and in the circumferential direction. Crossing can also occur at a particular distance apart.

In another advantageous configuration, the fibers extend in the longitudinal direction of the liner from the distal end to the proximal opening. This has the result that an elastic elongation of the liner in this direction cannot take place. Nevertheless, when it is rolled onto an amputation stump, such a liner is also capable of adapting to very different volumes and expansions of the particular amputation stump and to securely enclose the amputation stump. If such a liner has been rolled onto a narrow amputation stump, which consequently has only a small circumference, the proximal periphery which encloses the proximal opening of the liner is located further up on the amputation stump than if the same liner were to be pulled over a thick amputation stump, which consequently has a large circumference. In this case, too, a change in the amputation stump volume, for example an increase in the volume, has the result that the liner can only follow this change in volume when the proximal periphery of the liner slips on the amputation stump of the patient. However, this is associated with extensive slipping of the entire inner side of the liner on the amputation stump, this being prevented by the anti-slip means.

Alternatively, it is also possible for the plurality of fibers to form a net-like structure having a plurality of mutually adjoining meshes. Preferably, the meshes have a hexagonal shape. Such nets or net-like structures have the required properties and allow elastic deformation in the longitudinal direction and in the circumferential direction of the liner, as long as they are arranged in the correct orientation. These nets can also be prefabricated in an extensive manner and cut to size and embedded in the liner material during the production of the liner. As a result, the manufacturing process is simplified and streamlined and thus the production costs are lowered.

It is consequently important for the functioning of the liners described herein that the liner is secured to the amputation stump at the proximal periphery such that it cannot shift relative to the amputation stump. Consequently it is necessary to prevent, as effectively as possible, the liner from slipping downward on the amputation stump, it being possible for this to occur in particular during the course of a day in which the liner is worn for example over a long period of time. This can occur on the one hand in that the adhesive friction between the liner material and the skin of the amputation stump is increased. This is possible for example via a special coating on the inner side of the liner. Alternatively or in addition thereto, the anti-slip means described herein can also be used. These may be provided for example in the form of a binding which is arranged in the proximal region of the liner. In this case, a belt or fastening strap is placed around the amputation stump in the proximal region of the liner and fastened such that a pressure is exerted on the liner in this region, said pressure acting in the direction of the amputation stump. As a result, the adhesive friction of the liner against the skin of the amputation stump is greatly increased in this region and thus the liner is prevented from shifting relative to the stump.

Instead of a belt, use can also be made of what are known as climbing skins which are arranged on a separate anti-slip means which is arranged in the proximal region of the liner. This can take place for example via a separate apparatus which is arranged in the distal region of the liner and for example is connected to the liner in this distal region via a pin or some other fastening device. The climbing skin is in this way positioned in a fixed position in the distal region of the liner relative to the amputation stump and can prevent any movement of the liner in one direction via a direction of the fibers of the climbing skin, while it does not impair a movement in the opposite direction in each case or only impairs such a movement a little. In this way, too, the liner can be secured to the amputation stump in the proximal region.

At the distal end of the liner, the liner is advantageously held in position relative to the prosthesis socket. This can occur using all the methods that are known per se from the prior art, for example by a locking pin. As a result of the clear position of the liner ends fixed in this way, in combination with the particular use of the longitudinally stable fibers, the volume of the liner, said volume corresponding to the volume of the amputation stump when the liner is fitted, can be readily held and thus also the amputation stump can be kept constant in terms of volume.

A prosthesis according to the invention has an above-described liner, a prosthesis socket and a distal prosthetic device which is securable to a distal end of the prosthesis socket. In this case, the prosthesis socket preferably has a proximal socket region and a distal socket region which are connected together in a spaced-apart manner by a connecting device.

The prosthesis socket is consequently in the form of a frame-type socket, with the result that it has the above-mentioned advantages. The distal socket region is advantageously in the form of a bowl or cup and is designed to accommodate the distal end of the amputation stump. Via the proximal socket region, the prosthesis socket is secured to the amputation stump and fixed in a particular orientation. In this way, the patient is provided with a sufficiently stabilized connection to the prosthesis and the possibility of controlling the latter in an optimal manner. Such a prosthesis is in particular a transtibial prosthesis. Such a prosthesis is used when a leg of a patient has had to be amputated below the knee. The patient's remaining section of lower leg has in this case a greatly differing length from patient to patient. This length can be dealt with by way of the connecting device with which the proximal socket region is connected to the distal socket region, in that the connecting device is configured to have an adjustable length. The longer the connecting device can be configured to be, the further the proximal socket region and the distal socket region are separated from one another and the greater the control and controllability of the prosthesis by the patient are. At the same time, in this way, the interruptions in the frame-type socket formed in such a way are configured to be as large as possible so that the advantages thereof come into effect as comprehensively as possible.

The prosthesis socket can be secured to the liner in various forms. For example, a fastening device, for example in the form of a distally projecting pin, can be provided at a distal end of the liner, said fastening device being able to be inserted into a receiving device, provided for this purpose, on the prosthesis socket. Alternatively, a retaining force between the prosthesis socket and the liner can also be brought about via surface friction between the outer side of the liner material and the inner side of the prosthesis socket. Preferably, both effects are combined with one another in order to ensure that the prosthesis socket is retained on the liner as securely as possible and thus also on the amputation stump.

In a preferred embodiment, the proximal socket region has an opening for receiving the amputation stump, wherein a circumferential length of the opening is settable. This can be achieved for example by at least two socket elements which are adjustable with respect to one another via at least one adjusting element. This ensures that the size of the opening into which the amputation stump having the liner pulled over it is introduced is settable in an individual manner. As a result, use can be made of a standard socket which is adaptable to the individual circumstances of the amputation stump of the patient. The two socket elements can be formed for example in the form of tongue-like protrusions which are directed toward one another in the circumferential direction. These protrusions can be connected together via a belt or some other suitable connecting device, wherein the belt preferably has a variable length. In this way, the circumference of the proximal opening of the socket can be changed and adapted to the individual circumstances of the patient.

In particular, as a result of the selected configuration of the prosthesis, it is possible to combine different standard components with one another and in this way to achieve a prosthesis that is individually adapted in each case. In this way, it is for example possible to keep in stock different distal socket regions, that is to say for example cups with different sizes and depths, and different proximal socket regions and connecting devices with different lengths, these being configured to be combinable with one another in each case. Individual adaptation to the amputation stump of the patient is enabled in this way.

A prosthetic device, which can consist for example in the form of an artificial lower leg with a prosthetic foot located thereon, is securable to the distal and/or proximal end of the prosthesis socket. This prosthetic device, too, can be configured to be pivotable in a large variety of directions, in order to achieve the optimal position for the particular amputation stump of the patient. At the same time, the artificial lower leg has in particular an adjustable length in order to be able to react to amputation stumps with different lengths.

The invention additionally achieves the stated object by way of a prosthesis socket for such a prosthesis.

An exemplary embodiment of the present invention is explained in more detail in the following text with the aid of a drawing, in which

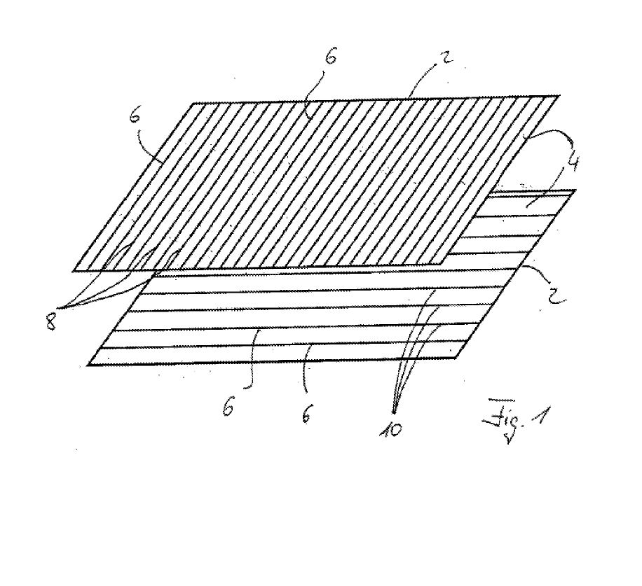

FIG. 1 shows the schematic arrangement of two layers of a liner material,

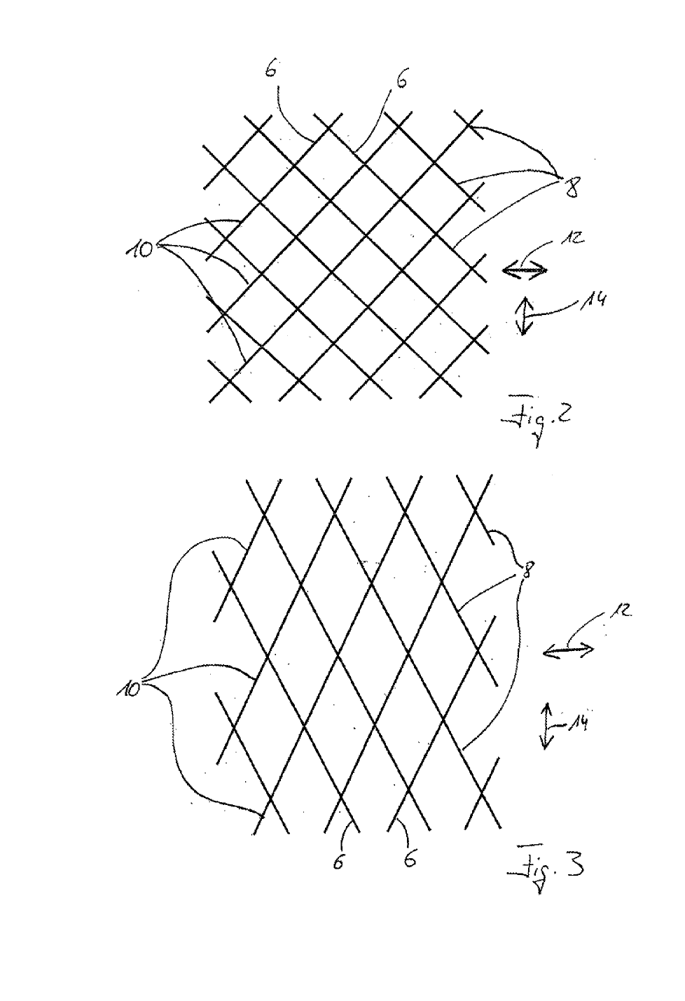

FIG. 2 shows an arrangement of a plurality of fibers in a first orientation,

FIG. 3 shows the arrangement of the plurality of fibers in a second orientation,

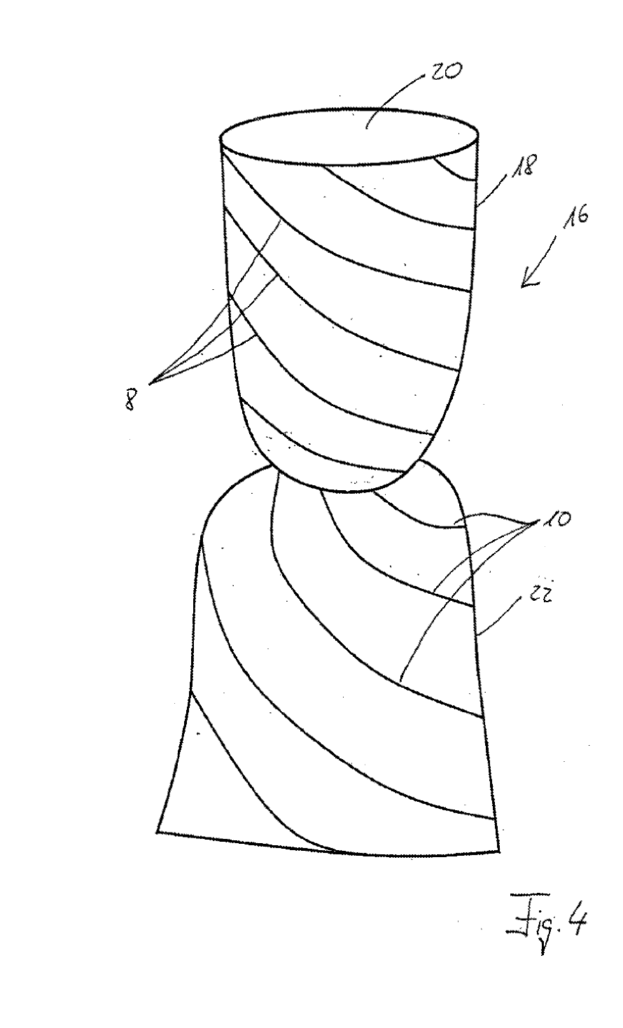

FIG. 4 shows the schematic illustration of a liner according to one exemplary embodiment of the present invention,

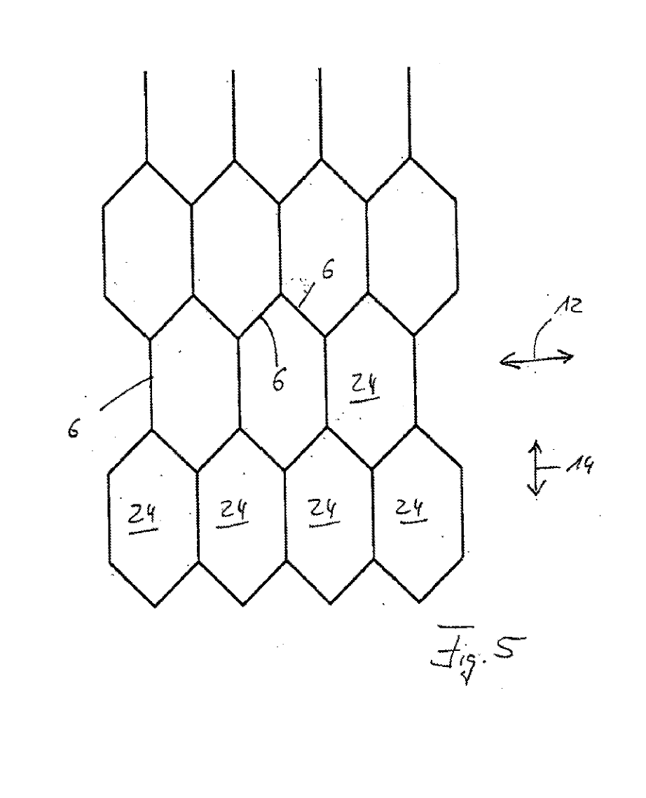

FIG. 5 shows a further possible arrangement of the fibers in a liner,

FIG. 6 shows the schematic illustration of an anti-slip means,

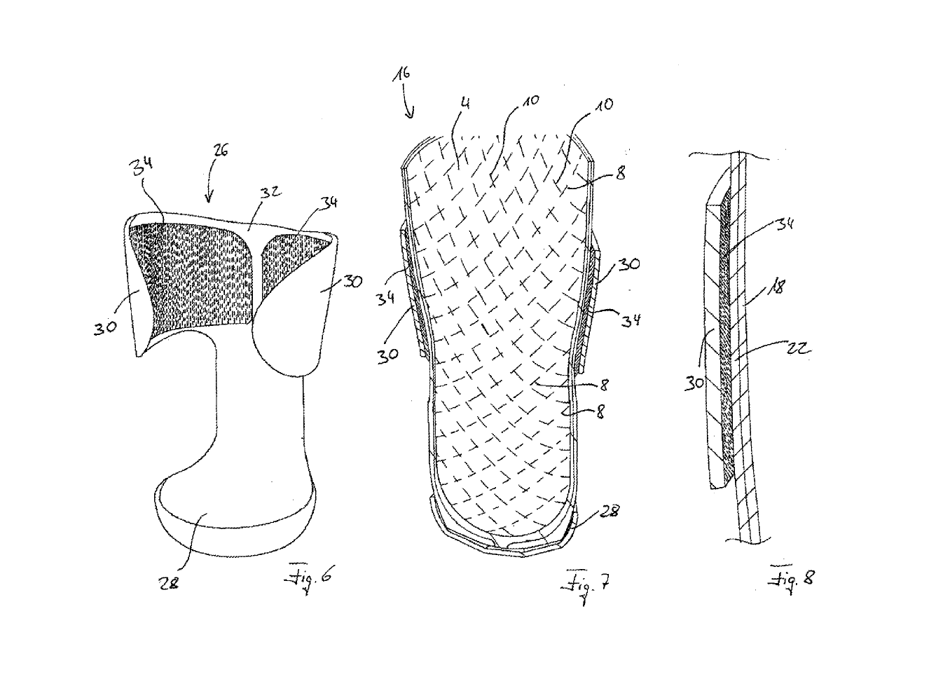

FIG. 7 shows a sectional illustration of the anti-slip means from FIG. 6 in the fitted state,

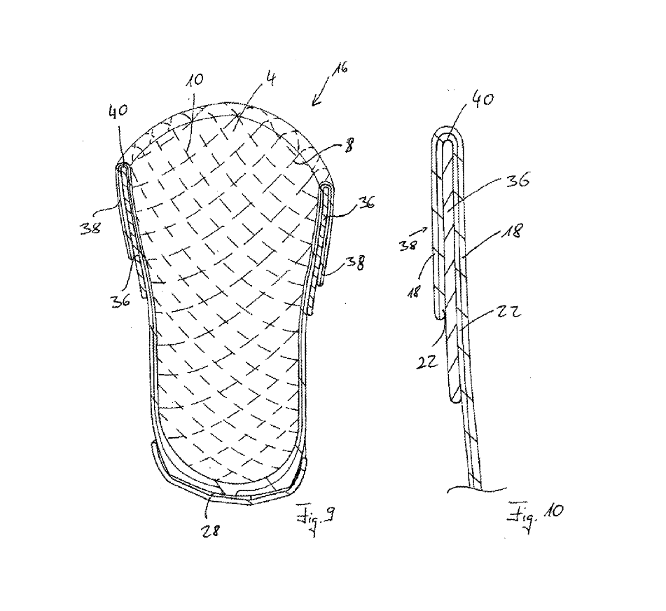

FIG. 8 shows an enlarged detail from FIG. 7,

FIG. 9 shows a schematic sectional illustration through a prosthesis according to a further exemplary embodiment of the present invention,

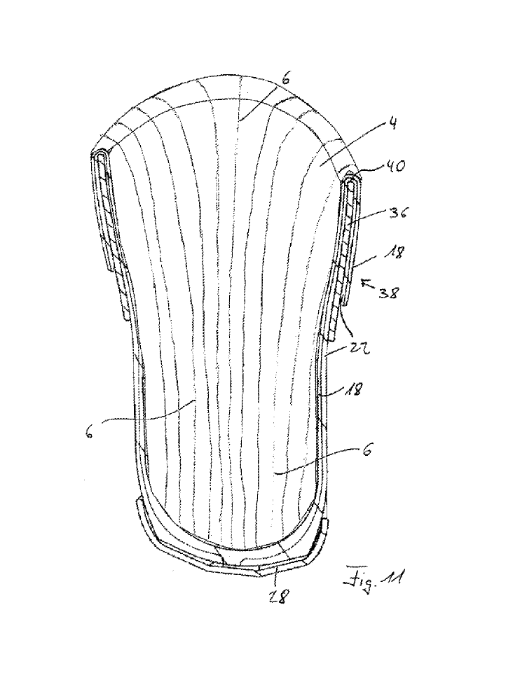

FIG. 10 shows an enlarged detail from FIG. 9,

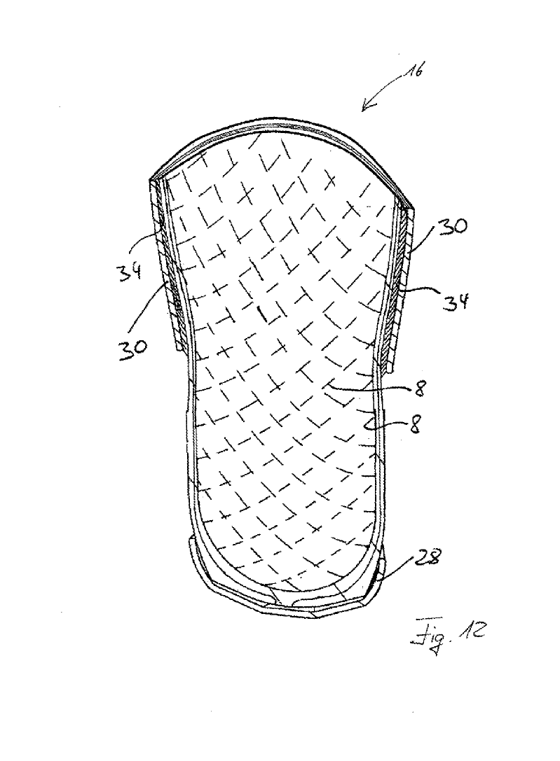

FIG. 11 shows the schematic sectional illustration through a further prosthesis according to one exemplary embodiment of the present invention,

FIG. 12 shows the schematic sectional illustration through a further prosthesis according to one exemplary embodiment of the present invention.

FIG. 1 schematically shows two layers 2 of a liner material 4. Each layer 2 has a plurality of fibers 6 which are arranged parallel to one another in the respective layer 2. However, between the individual layers 2, the fibers 6 are offset with respect to one another through 90 degrees. All of the fibers 6 in the upper layer 2 are in this case a first fiber layer 8, while all of the fibers 6 in the lower layer 2 form a second fiber layer 10. In the fitted state of a liner which comprises the two layers 2, this arrangement of the fibers 6 in the first fiber layer 8 and the second fiber layer 10 has the result that elastic deformation of the liner in one direction results in elastic deformation in a second direction that extends perpendicularly thereto.

FIG. 2 shows the first fiber layer 8 and the second fiber layer 10 in a liner 16 according to one exemplary embodiment of the present invention. In this case, it is immaterial whether the first fiber layer 8 and the second fiber layer 10 are arranged in one layer 2 or in two separate layers 2 which are pulled over one another to form a liner. It can be seen that the fibers 6 of the first fiber layer 8 and the fibers 6 of the second fiber layer 10 each enclose a right angle with one another. An elongation of the liner in a first direction, which is illustrated by the arrow 12 in FIG. 2, consequently results in shortening of the liner 16 in a second direction extending perpendicularly thereto, this second direction being illustrated by the arrow 14. This can be achieved in particular in that the individual fibers 6 are formed in a nonelastic manner or at least a less elastic manner than the liner material 4 in which they are embedded. It is thus not possible or only possible to a very small degree to stretch the individual fibers 6 along their longitudinal extent, and so elongation of the liner in the first direction cannot be achieved or can only be achieved to a very small degree by the individual fibers 6 being stretched.

FIG. 3 shows the first fiber layer 8 and the second fiber layer 10 from FIG. 2, wherein the liner has now been stretched in the direction of the second direction, which is illustrated by the arrow 14. It can be seen that the fibers 6 of the first fiber layer 8 and of the second fiber layer 10 no longer enclose a right angle, but stretched rhombuses are now formed between the individual fibers. The extension and elongation of the liner in the first direction of the arrow 14 simultaneously results in shortening of the liner in the second direction, opposite thereto, which is indicated by the arrow 12. In the fitted state of the line 16, such a shortening can only occur when the liner slides extensively along the amputation stump. Since, however, the adhesion between the liner material 4 and amputation stump is relatively high, such sliding along is not possible. Consequently, once it is in the fitted state, the liner 16 cannot follow a change in volume of the amputation stump or can follow this only to a very small degree, with the result that there is volume stability.

FIG. 4 shows a particular configuration of a liner 16. It consists of a first part-liner 18 which has an opening 20 at its proximal end. The amputation stump is introduced into the latter. The first part-liner 18 has the first fiber layer 8, which in this case forms a spiral matrix which has a first circumferential direction.

Arranged at the distal end of the first part-liner 18 is a second part-liner 22 in which the second fiber layer 10 is located. This, too, is a spiral matrix, but extending in the opposite direction. In order to fit the liner 16 on an amputation stump (not shown), the second part-liner 22 is rolled over the first part-liner 18 such that both the first part-liner 18 and the second part-liner 22 envelop the amputation stump and the first fiber layer 8 and the second fiber layer 10 are arranged one over the other. Since both fiber layers 8, 10 form spiral matrices, but with different circumferential directions, the individual fibers 6 of the two fiber layers 8, 10 overall form a net-like structure which has precisely the desired properties.

FIG. 5 shows a further possible arrangement of the different fibers 6, which in this case form a net-like structure having mutually adjoining meshes 24. Each of these meshes 24 has a hexagonal shape in the exemplary embodiment shown in FIG. 5. As a result of this shape of the net-like structure, in this arrangement of the fibers, too, an elongation of the liner in the direction of the arrow 12 results in shortening of the liner in the direction of the arrow 14 and vice versa, and so this arrangement, too, has the desired effect according to the invention.

FIG. 6 shows an anti-slip means 26 as can be used in a prosthesis according to one exemplary embodiment of the present invention. The anti-slip means 26 is configured as a separate component but is nevertheless considered to be a part of the liner. It comprises in its distal region a receiving cup 28 into which the distal end of the amputation stump is inserted. Arranged in the proximal region of the anti-slip means 26 are two tongue-like protrusions 30 which at least partially enclose the amputation stump with the liner material located around it. Arranged on an inner side 32 are climbing skins 34 by way of which a relative movement of the liner material with respect to the tongue-like protrusions 30 is prevented at least in one direction.

FIG. 7 shows a sectional illustration through a liner 16 having an anti-slip means 26. Arranged in the liner material 4, the first fiber layer 8 and the second fiber layer 10, which are arranged such that the individual fibers cross, can be seen. The receiving cup 28 can be seen in the distal region while the two tongue-like protrusions 30 are illustrated in the proximal region of the anti-slip means 26, wherein the climbing skins 34 are located on the inner side 32 of said protrusions 30. This part is illustrated in an enlarged manner in FIG. 8. The first part-liner 18 and the second part-liner 22, which rest against one another, can be seen. Located on the outer side of the second part-liner 22 is a tongue-like protrusion 30 with the climbing skin 34 arranged on its inner side. In the case of the shown arrangement of the climbing skin 34, the individual hairs or bristles of the climbing skin 34 are directed downwardly from the tongue-like protrusion 30, and so it is possible for the liner material 4 to shift and slip downward relative to the tongue-like protrusions 30, while the liner material 4 is prevented from shifting upward. Such an arrangement is consequently sensible in particular for the case in which the volume of the amputation stump is expected to decrease during the course of a day. This would have the consequence of the liner material 4 shifting upward, since a decrease in the extension of the liner 16 in the circumferential direction would result in elongation in the longitudinal direction. However, this shifting is prevented by the climbing skins 34 on the inner side of the tongue-like protrusions 30.

FIG. 9 shows another configuration of a liner 16. Here, too, the first fiber layer 8 and the second fiber layer 10, which are embedded in the liner material 4, are illustrated. The receiving cup 28 of the anti-slip means is again arranged in the distal region of the liner 16. Of course, anti-slip means that do not have a receiving cup 28 also exist. Located in the proximal region of the anti-slip means is an enclosing element 36, which completely encloses the amputation stump in the exemplary embodiment shown. It can be manufactured for example from an elastic material and, in this case, when the amputation stump is arranged within the enclosing element 36, is stretched such that it exerts a force in the radial direction with respect to the longitudinal direction of the amputation stump. In this way, a greater force is applied to the liner material 4, and thus also to the amputation stump located therein, in the region in which the enclosing element 36 is located, and so increased adhesive friction is achieved.

In FIG. 10, it can be seen that a proximal region 38 of the liner material 4 has been folded over a top edge of the enclosing element 36. This situation is illustrated in an enlarged manner in FIG. 10. The folding over of the proximal region 38 of the first part-liner 18 and of the second part-liner 22 has a number of effects. Firstly, the enclosing element 36 is enclosed between two layers of the liner material. This is sensible in particular for the case in which the anti-slip means 26 consists only of the enclosing element 36 and does not also have a receiving cup 28 or some other element by way of which optimal and fixed positioning of the enclosing means 38 on the amputation stump is ensured. In addition, as a result of the folded-over proximal region 38 of the liner material 4, a further force is exerted in the radial direction on the enclosing element and thus also on the amputation stump located therein, and so a further increase in the adhesive friction is achieved.

FIG. 11 shows an illustration similar to FIG. 9, in which provision is likewise made of an enclosing element 36, which has been placed about an amputation stump (not shown). A proximal region 38 of the liner material 4 has been flapped over the top edge 40 of the enclosing element 36. Unlike the liner shown in FIG. 9, the liner shown in FIG. 11 has only one fiber layer, which consists of fibers 6 which extend only in the longitudinal direction of the liner, that is to say from the distal end of the liner to the proximal periphery of the liner. This has the result that elastic deformation in the longitudinal direction is no longer possible, although the liner, when pulled and rolled onto an amputation stump, can adapt optimally to the shape of the amputation stump.

The liner 16 can in this case advantageously have in its distal region a fastening element which can be configured for example as a pin or lock or similar apparatus. By way of this apparatus, it is possible to fasten the distal region of the liner to the receiving cup 28 and in this way to achieve a particularly stable connection between the liner 16 and the receiving cup 28, or a prosthesis socket fastened thereto. The volume of the liner 16 which is filled by the amputation stump is in this way determined by an effective fiber length of the fibers 6, said fiber length being defined from the distal fastening of the liner to the receiving cup 28 to the proximal end of the liner by way of which it is secured to the amputation stump, and by the contour of the amputation stump.

In general, however, the effective weight force acts for example in the standing phase in such a way that the stump bears against the receiving cup 28, or a corresponding distal socket bowl, and cannot slip out in the proximal direction. This is important because as a result the liner is prevented from taking up the maximum possible volume which is allowed by the introduced fibers 6. This would result in the volume changing while the liner is being worn, such that the desired volume stability is no longer provided. Therefore, it is essential for the functioning of the liner described here that the liner cannot slip relative to the amputation stump but is secured in particular in the proximal region.

This can occur using the anti-slip means shown here, for example a binding or the climbing skins 34 shown. However, it may also be sufficient to achieve this merely by way of a coating applied for example to the inner side of the liner, said coating increasing adhesive friction between the liner and the skin of the amputation stump.

FIG. 12 shows a sectional illustration, similar to FIG. 7, through a liner 16 having an anti-slip means 26. Unlike the liner 16 shown in FIG. 7, the liner ends flush with the tongue-like protrusions 30 of the anti-slip means 26 in the proximal region, that is to say at the top in FIG. 12. This is an advantageous but in no way necessary configuration of the liner. While the liner 16 shown in FIG. 7 can be folded over, as illustrated in FIG. 9, and thus at least partially envelops the anti-slip means 26 on both sides, that is to say from the inside and from the outside, this is not possible in the case of a liner 16 according to the embodiment shown in FIG. 12. However, here too, climbing skins 34 are arranged on the inner side 32 of the anti-slip means 26, said climbing skins 34 preventing a relative movement of the liner material with respect to the tongue-like protrusions 30 at least in one direction. For most applications it is sufficient to provide the climbing skins 34 as a single component, said climbing skins 34 bringing about the effect of the anti-slip means 26. Flapping over the liner 16, as illustrated in FIG. 9, in order to apply further pressure from the outer side of the tongue-like protrusions 30 and thus to enhance the effect of the climbing skins 34 is advantageous but not necessary.

1-14. (canceled)

15. A prosthesis, comprising:
a prosthetic socket having a proximal region and a distal region, the distal region and the proximal region configured to secure the prosthetic socket to the residual limb in a fixed orientation;
a liner configured to receive a residual limb and to be inserted with the residual limb into the prosthetic socket, the liner being secured to the residual limb at least in part by forces applied by the prosthetic socket, the liner comprising:
a liner body comprising:
a proximal opening to receive the residual limb;
a closed distal end opposite the proximal opening;
a longitudinal dimension extending from the proximal opening to the closed distal end;
a circumferential dimension extending perpendicular to the longitudinal dimension;
a plurality of fibers comprising a nonelastic material and being arranged such that an elongation of the liner material in the longitudinal dimension results in shortening of the liner material in the circumferential dimension and vice versa.

16. The liner as claimed in claim 15, further comprising an anti-slip component operable to prevent the liner from slipping on the residual limb.

17. The liner as claimed in claim 16, wherein the anti-slip component includes a surface property that engages an outer side of the liner body, an inner side of the liner body configured to rest against the residual limb.

18. The liner as claimed in claim 16, wherein the anti-slip component includes an enclosing element that at least partially encloses the liner material, the enclosing element being configured to prevent the liner material from slipping relative to the residual limb.

19. The liner as claimed in claim 15, wherein the plurality of fibers include a first fiber and a second fiber that cross each other at a right angle.

20. The liner as claimed in claim 15, wherein the plurality of fibers include a first fiber and a second fiber, the first fiber forms a spiral matrix in a first circumferential direction and the second fiber forms a spiral matrix in a second circumferential direction opposite to the first circumferential direction.

21. The liner as claimed in claim 15, wherein the plurality of fibers include a first fiber and a second fiber, the first fiber is part of a first fiber layer comprising a plurality of first fibers and the second fiber is part of a second fiber layer comprising a plurality of second fibers.

22. The liner as claimed in claim 15, wherein the plurality of fibers include a first fiber and a second fiber, the liner body has at least first and second part-liners, the first fiber being embedded in the first part-liner and the second fiber being embedded in the second part-liner, and the second part-liner is configured to be pulled over the residual limb after the first part-liner has been pulled over the amputation stump.

23. The liner as claimed in claim 15, wherein the plurality of fibers extend at least partially in the longitudinal dimension.

24. The liner as claimed in claim 15, wherein the plurality of fibers forms a net-like structure having a plurality of mutually adjoining meshes.

25. The liner as claimed in claim 24, wherein the mutually adjoining meshes have a hexagonal shape.

26. The liner as claimed in claim 15, wherein the prosthetic socket only partially encloses and surrounds the liner.

27. The liner as claimed in claim 15, wherein a distal end of the liner is secured to the socket.

28. The liner as claimed in claim 15, wherein the liner is secured to the residual limb at least at the distal and proximal ends of the liner.

29. A prosthesis, comprising:
a socket having a socket cavity, a distal region and a proximal region, the distal region and the proximal region configured to secure the prosthetic socket to the residual limb in a fixed orientation;
a liner configured to mount to the residual limb and be inserted with the residual limb into the socket cavity, a force to secure the liner to the residual limb being provided at least in part by the prosthetic socket, the liner comprising:
a liner body having a proximal opening to receive the residual limb, a closed distal end opposite the proximal opening, and a plurality of fibers which each comprise a nonelastic material and are arranged such that an elongation of the liner body in a longitudinal direction results in shortening of the liner body in a circumferential direction, wherein the longitudinal direction extends from the proximal opening to the closed distal end, and the circumferential direction extends perpendicular to the longitudinal direction.

30. The prosthesis as claimed in claim 29, further comprising an anti-slip component operable to prevent the liner from slipping on the residual limb.

31. The prosthesis as claimed in claim 30, wherein the anti-slip component includes a surface property that engages an outer side of the liner body, an inner side of the liner body configured to rest against the amputation stump, and the anti-slip component is configured to prevent the liner from slipping relative to the amputation stump.

32. The prosthesis as claimed in claim 29, wherein the plurality of fibers includes a first fiber and a second fiber which are arranged such that the first and second fibers cross one another.

33. The prosthesis as claimed in claim 29, wherein the plurality of fibers includes a first fiber and a second fiber, the first fiber forms a spiral matrix in a first circumferential direction and the second fiber forms a spiral matrix in a second circumferential direction opposite to the first circumferential direction.

34. The prosthesis as claimed in claim 29, wherein the plurality of fibers includes a first fiber and a second fiber, the first fiber is part of a first fiber layer comprising a plurality of first fibers and the second fiber is part of a second fiber layer comprising a plurality of second fibers.

35. The prosthesis as claimed in claim 29, wherein the plurality of fibers includes a first fiber and a second fiber, the liner body comprises first and second part-liners, the first fiber is embedded in the first part-liner and the second fiber is embedded in the second part-liner, and the second part-liner is configured to be positioned over the amputation stump after the first part-liner has been pulled over the amputation stump.

36. The prosthesis as claimed in claim 29, wherein the anti-slip component includes a cup-shaped distal end or a closed distal end.

37. The prosthesis as claimed in claim 29, wherein the anti-slip component includes an elongate tongue member that is arranged in the circumferential direction, the tongue member being spaced proximal of a distal end of the anti-slip component.

38. The prosthesis as claimed in claim 26, wherein the anti-slip feature is configured to be positioned in its entirety within the socket.
